(19) United States
(12) Patent Application Publication
(10) Pub. No.: US 2024/0238347 A1
(43) Pub. Date: Jul. 18, 2024

(54) PHOTORECEPTOR CELLS FOR RETINAL AND MACULAR REPAIR

(71) Applicant: The Johns Hopkins University, Baltimore, MD (US)

(72) Inventors: Mandeep Singh, Baltimore, MD (US); Robert Johnston, Baltimore, MD (US); Kiara Eldred, Baltimore, MD (US); Katarzyna Hussey, Baltimore, MD (US); Sarah Hadyniak, Baltimore, MD (US); Christina McNerney, Baltimore, MD (US)

(73) Assignee: The Johns Hopkins University, Baltimore, MD (US)

(21) Appl. No.: 18/581,734

(22) Filed: Feb. 20, 2024

Related U.S. Application Data
(63) Continuation of application No. PCT/US22/40853, filed on Aug. 19, 2022.
(60) Provisional application No. 63/235,380, filed on Aug. 20, 2021.

(51) Int. Cl. A61K 35/30; A61P 27/02

(52) U.S. Cl. CPC A61K 35/30; A61P 27/02

(57) ABSTRACT

The present disclosure relates to cell-based therapeutics for retinal and macular repair comprising defined retinal cell subtypes, including short (s)-, medium (m)- and long (l) wavelength sensitive cone photoreceptors, methods for the manufacture of such cell-based therapeutics, and methods of using such therapeutics.

### CROSS-REFERENCE TO RELATED APPLICATIONS

This application is a Continuation application of International Application No. PCT/US2022/040853, filed on Aug. 19, 2022, which claims the benefit of U.S. Provisional Application No. 63/235,380, filed on Aug. 20, 2021, the contents of which are incorporated by reference in their entirety, and to which priority is claimed.

### STATEMENT REGARDING FEDERALLY SPONSORED RESEARCH

This invention was made with government support under grant EY030872 awarded by National Institutes of Health. The government has certain rights in the invention.

### 1. FIELD OF INVENTION

The present disclosure relates to cell-based therapeutics for retinal and macular repair comprising defined retinal cell subtypes, including rod photoreceptors, short (S)-, medium (M)-, and long (L) wavelength sensitive cone photoreceptors, and related retinal interneurons, methods for the manufacture of such cell-based therapeutics, and methods of using such therapeutics.

### 2. BACKGROUND

Degenerative macular diseases are a major cause of incurable blindness. Examples include age related macular degeneration (AMD), Stargardt disease, cone dystrophy, achromatopsia, Best disease, mitochondrial macular degeneration, pattern dystrophy, RDS-associated macular degeneration, and other forms of inherited macular dystrophy. The macula enables high-acuity vision due to its high density of cone photoreceptors. Furthermore, the ratio of rods, S, M, and L cones in the normal macula is a key determinant of normal high-acuity vision. The main cellular cause of morbidity in degenerative macular diseases is the degeneration of cone photoreceptor cells in the human macula, which results in the diminution of high-acuity, central, and bright-light vision that is required for face recognition and reading.

Currently there are limited options for patients diagnosed with degenerative macular diseases. In addition, such options typically reduce the rate of degeneration rather than offer an avenue for actual repair of diseased tissue. Accordingly, there remains a need in the art for additional therapeutics for addressing degenerative macular diseases, particularly those that can provide for repair of diseased tissue.

### 3. SUMMARY

In certain embodiments, the present disclosure is directed to cell-based therapeutics, e.g., cellular compositions, for retinal and macular repair comprising defined retinal cell subtypes, including rod photoreceptors, short (S)-, medium (M)-, and long (L) wavelength sensitive cone photoreceptors, and related retinal interneurons, methods for the manufacture of such cell-based therapeutics, and methods of using such therapeutics.

In certain embodiments, the present disclosure is directed to cellular composition comprising a population of L cones and M cones, wherein the ratio of L cones to M cones (L:M) falls within a predetermined range. In certain embodiments, the predetermined L:M range is from about 1.3:1 to about 2.8:1. In certain embodiments, the predetermined L:M ratio is about 2:1. In certain embodiments, the population of cones comprises up to about 2% S cones as a percentage of total cones. In certain embodiments, the population of cones comprises up to about 5% S cones as a percentage of total cones.

In certain embodiments, the ratio of the population of S, M, and L cones (S:M:L) falls within a predetermined range. In certain embodiments, the S:M:L is about 1:33:66. In certain embodiments, the S:M:L is about 3:33:64. In certain embodiments, the predetermined S:M:L ratio is a ratio that occurs naturally in trichromatic patients with normal color vision at a particular retinal eccentricity. In certain embodiments, the particular retinal eccentricity is at or around the foveal umbo. In certain embodiments, the particular retinal eccentricity inner boundary is at about 0 mm and the outer boundary is at about 0.1 mm linear eccentricity. In certain embodiments, the particular retinal eccentricity is at or around the foveal center. In certain embodiments, the particular retinal eccentricity inner boundary is at about 0.1 mm and the outer boundary is at about 0.175 mm linear eccentricity.

In certain embodiments, the composition comprises rods (“R”) and wherein the S:M:L:R ratio falls within a predetermined range. In certain embodiments, the predetermined S:M:L:R ratio is a ratio that occurs naturally in trichromatic patients with normal color vision at a particular retinal eccentricity. In certain embodiments, the particular retinal eccentricity is at or around the foveal center. In certain embodiments, the particular retinal eccentricity inner boundary is at about 0.175 mm and the outer boundary is at about 0.750 mm linear eccentricity. In certain embodiments, the population of cones comprises about 10% to about 20% S cones as a percentage of total cones. In certain embodiments, the population of rods comprises about 55% to about 80% rods as a percentage of total cones and rods combined. In certain embodiments, the ratio of S:M:L:R is about 6:11:23:60. In certain embodiments, the particular retinal eccentricity is at or around the parafovea. In certain embodiments, the particular retinal eccentricity inner boundary is at about 0.750 mm and the outer boundary is at about 1.50 mm linear eccentricity. In certain embodiments, the population of cones comprises about 7% to about 10% S cones as a percentage of total cones. In certain embodiments, the population of rods comprises about 60% to about 95% rods as a percentage of total cones and rods combined. In certain embodiments, the ratio of S:M:L:R is about 2:6:12:80. In certain embodiments, the particular retinal eccentricity is at or around the perifovea. In certain embodiments, the particular retinal eccentricity inner boundary is at about 1.50 mm and the outer boundary is at about 3.0 mm linear eccentricity. In certain embodiments, the population of cones comprises about 6% to about 9% S cones as a percentage of total cones. In certain embodiments, the population of rods comprises about 75% to about 95% rods as a percentage of total cones and rods combined. In certain embodiments, the ratio of S:M:L:R is about 1:3:7:90. In certain embodiments, the particular retinal eccentricity is at or around the peripheral macula. In certain embodiments, the particular retinal eccentricity inner boundary is at about 3.0 mm and the outer boundary is at about 4.5 mm linear eccentricity. In certain embodiments, the population of rods comprises about 55% to about 85% rods as a percentage of total cones and rods combined. In certain embodiments, the ratio of S:M:L:R is about 2:9:19:70. In certain embodiments, the particular retinal eccentricity is at or around the pericentric retina. In certain embodiments, the particular retinal eccentricity inner boundary is at about 4.50 mm and the outer boundary is at about 6.0 mm linear eccentricity. In certain embodiments, the population of rods comprises about 50% to about 80% rods as a percentage of total cones and rods combined. In certain embodiments, the ratio of S:M:L:R is about 2:11:22:65. In certain embodiments, the particular retinal eccentricity is at or around the peripheral retina. In certain embodiments, the particular retinal eccentricity inner boundary is at about 6.0 mm and the outer boundary is at about 7.5 mm linear eccentricity. In certain embodiments, the ratio of S:M:L:R is about 3:10:22:65. In certain embodiments, the particular retinal eccentricity is at or around the far peripheral retina. In certain embodiments, the particular retinal eccentricity inner boundary is at about 7.50 mm and the outer boundary is greater than about 7.50 mm linear eccentricity. In certain embodiments, the population of rods comprises about 60% to about 90% rods as a percentage of total cones and rods combined. In certain embodiments, the ratio of S:M:L:R is about 2:7:14:75.

In certain embodiments, the present disclosure is directed to compositions comprising two regions, wherein each region comprises a different M:L ratio. In certain embodiments, the compositions comprise two regions, wherein each region comprises a different S:M:L ratio. In certain embodiments, the compositions comprise two regions, wherein each region comprises a different S:M:L:R ratio.

In certain embodiments, the present disclosure is directed to cellular compositions comprising a population of cones, wherein one or more of the cone outer segments exhibits a capacitance, and wherein the composition is characterized by membrane currents in the range of 500-2500 pA.

In certain embodiments, the present disclosure is directed to methods for preparing cellular compositions comprising a predetermined population of S-, M-, and L-cones where the methods comprise culturing an organoid such that the organoid achieves the desired S-, M-, and L-cone ratios.

In certain embodiments, the present disclosure is directed to methods for preparing cellular compositions comprising a predetermined population of S-, M-, and L-cones wherein the methods comprise: a) culturing two or more independent organoids; and b) combining cells obtained from the two or more independent organoids to achieve the desired S-, M-, and L-cone ratios.

In certain embodiments, the present disclosure is directed to methods of treating age-related macular degeneration comprising engraftment into the macula of a patient in need thereof of a cellular composition as described herein.

In certain embodiments, the present disclosure is directed to methods of treating retinal degeneration comprising engraftment into the macula of a patient in need thereof of a cellular composition as described herein.

In certain embodiments, the retinal degeneration treated by the methods of the present disclosure is due to age-related macular degeneration, Stargardt disease, cone dystrophy, achromatopsia, Best disease, mitochondrial macular degeneration, pattern dystrophy, or RDS-associated macular degeneration.

### 4. BRIEF DESCRIPTION OF THE DRAWINGS

FIG. 1 illustrates the different anatomic zones of the macula and retina, which can be used to inform macular repair cell designs to regenerate the approximate cellular composition that is found at zones in normal (trichromatic) subjects.

FIG. 2 provides macular repair cell designs for specific macular and retinal zones.

FIG. 3 provides specification ranges for particular macular repair cell designs.

FIG. 4 depicts the temporal variation in the ratio of photoreceptor identity in an exemplary culture lacking retinoic acid (RA).

### 5. DETAILED DESCRIPTION

The present disclosure relates to cell-based therapeutics for retinal and macular repair comprising defined retinal cell subtypes, including rods and short (S)-, medium (M)-, and long (L) wavelength sensitive cone photoreceptors, methods for the manufacture of such cell-based therapeutics, and methods of use of such therapeutics. The cell-based therapeutics of the present disclosure are referred herein as macular repair cells (MARC).

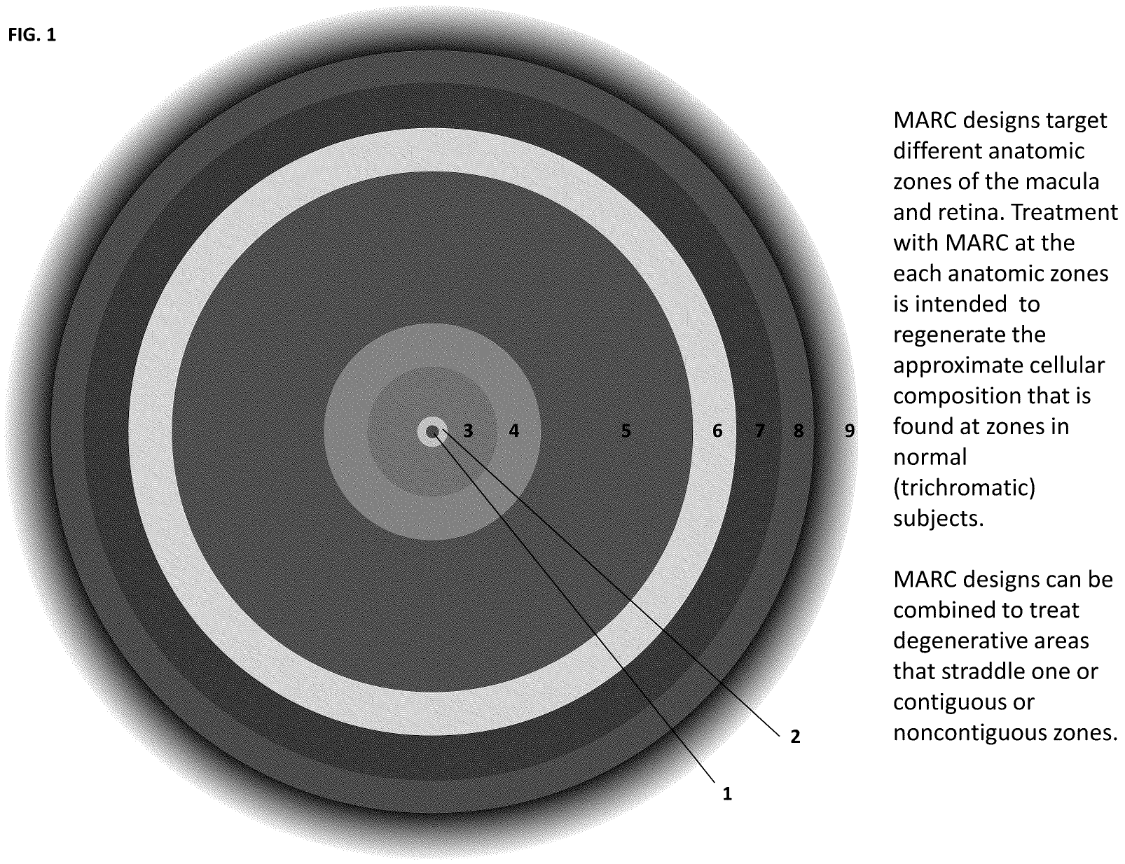

In one aspect, the subject matter of the present disclosure is directed to MARC therapeutic compositions. For example, but not limitation, the present disclosure is directed to MARC compositions comprising a population of cone cells, wherein the ratio of S (“blue”), M (“green”), and L (“red”) cones falls within particular ranges. Design variations in MARC are created such that regenerative substrates can be designed to target degenerative loci at different antero-posterior eccentricities within the retina, as illustrated in FIG. 1. The MARC variation targeting each target location is designed to mimic the short (S)-, medium (M)-, long (L) wavelength sensitive cone photoreceptors, as well as rod (R) photoreceptors ratios (S:M:L:R ratio) that occurs naturally in trichromatic patients with normal color vision at that eccentricity.

In another aspect, the present disclosure is directed to methods for the manufacture of MARC therapeutics. In certain embodiments, MARC therapeutics can be generated via human retinal organoid culture. For example, but not limitation, MARC therapeutics can be generated using unique stem cell based human retinal organoid protocols that specifically enrich for S, M, and/or L cones. By enriching for specific ratios of S, M, and/or L cones, the methods of manufacture described herein can produce MARC therapeutics designed to regenerate the approximate cellular composition that is found at specific zones (or across specific zones) in normal trichromatic subjects.

In another aspect, the subject matter of the present disclosure is directed to macular regenerative therapy by MARC transplantation as a treatment to ameliorate functional deficits in people with retinal degenerative diseases. For example, but not limitation, retinal degenerative diseases that can be ameliorated by administration of the MARC compositions of the present disclosure include age-related macular degeneration, Stargardt disease, cone dystrophy, achromatopsia, Best disease, mitochondrial macular degeneration, pattern dystrophy, RDS-associated macular degeneration, and other forms of inherited macular dystrophy. As described herein, MARC therapeutics can be transplanted into the macula, and such transplantation can lead to regeneration of cone photoreceptor cells. Not only can MARC transplantation preserve and/or improve vision in people affected by macular diseases, but MARC delivery, as described in detail herein, is performed to ensure optimal maturation and integration of MARC therapeutics into the macula.

For clarity, but not by way of limitation, the detailed description of the presently disclosed subject matter is divided into the following subsections:

5.1 Definitions

5.2 Design Variation in Macular Repair Cell Compositions

5.3 Methods of Producing Macular Repair Cell Compositions

5.4 Methods of Treatment Using Macular Repair Cell Compositions

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

### 5.2. Design Variation in Macular Repair Cell Compositions

In one aspect, the subject matter of the present disclosure is directed to MARC therapeutic compositions. For example, but not limitation, the present disclosure is directed to MARC compositions comprising a population of cone cells, wherein the ratio of S, M, and L cones falls within particular ranges. Design variations in MARC are created such that regenerative substrates can be designed to target degenerative loci at different antero-posterior eccentricities within the retina, as illustrated in FIG. 1. The MARC variation targeting each target location is designed to mimic the S:M:L:R ratio that occurs naturally in trichromatic patients with normal color vision at that eccentricity.

In certain embodiments, the MARC composition of the present disclosure is a “MARC1” composition. MARC1 compositions are designed for foveal center regeneration at or around the foveal umbo at about 0 mm and up to about 0.1 mm linear eccentricity of the inner and outer boundaries, respectively. In certain MARC1 compositions, the L:M ratio ranges from about 1.3:1 to about 2.8:1. In certain embodiments, the MARC1 L:M ratio is about 2:1. In certain embodiments, MARC1 compositions comprise up to about 2% S-cones (as a percentage of total cones). In certain embodiments, MARC1 compositions contain no rods. In certain embodiments, MARC1 compositions have a S:M:L:R ratio that is about 1:33:66:0.

In certain embodiments, the MARC composition of the present disclosure is a “MARC2” composition. MARC2 compositions are designed for foveal center regeneration at or around the foveola at about 0.1 mm and up to about 0.175 mm linear eccentricity of the inner and outer boundaries, respectively. In certain MARC2 compositions, the L:M ratio ranges from about 1.3:1 to about 2.8:1. In certain embodiments, the MARC2 L:M ratio is about 2:1. In certain embodiments, MARC2 compositions comprise up to about 5% S-cones (as a percentage of total cones). In certain embodiments, MARC2 compositions contain no rods. In certain embodiments, MARC2 compositions have a S:M:L:R ratio that is about 3:33:64:0

In certain embodiments, the MARC composition of the present disclosure is a “MARC3” composition. MARC3 compositions are designed for foveal center regeneration at or around the fovea at about 0.175 mm and up to about 0.750 mm linear eccentricity of the inner and outer boundaries, respectively. In certain MARC3 compositions, the L:M ratio ranges from about 1.3:1 to about 2.8:1. In certain embodiments, the MARC3 L:M ratio is about 2:1. In certain embodiments, MARC3 compositions comprise about 10% to about 20% S-cones (as a percentage of total cones). In certain embodiments, MARC3 compositions comprise about 55% to about 80% rods (as a percentage of total cones+rods). In certain embodiments, MARC3 compositions have a S:M:L:R ratio that is about 6:11:23:60.

In certain embodiments, the MARC composition of the present disclosure is a “MARC4” composition. MARC4 compositions are designed for regeneration at or around the parafovea at about 0.750 mm and up to about 1.5 mm linear eccentricity of the inner and outer boundaries, respectively. In certain MARC4 compositions, the L:M ratio ranges from about 1.3:1 to about 2.8:1. In certain embodiments, the MARC4 L:M ratio is about 2:1. In certain embodiments, MARC4 compositions comprise about 7% to about 10% S-cones cones (as a percentage of total cones). In certain embodiments, MARC4 compositions comprise about 60% to about 95% rods (as a percentage of total cones+rods). In certain embodiments, MARC4 compositions have a S:M:L:R ratio that is about 2:6:12:80.

In certain embodiments, the MARC composition of the present disclosure is a “MARC5” composition. MARC5 compositions are designed for regeneration at or around the perifovea at about 1.5 mm and up to about 3.0 mm linear eccentricity of the inner and outer boundaries, respectively. In certain MARC5 compositions, the L:M ratio ranges from about 1.3:1 to about 2.8:1. In certain embodiments, the MARC5 L:M ratio is about 2:1. In certain embodiments, MARC5 compositions comprise about 6% to about 9% S-cones (as a percentage of total cones). In certain embodiments, MARC5 compositions comprise about 75% to about 95% rods (as a percentage of total cones+rods). In certain embodiments, MARC5 compositions have a S:M:L:R ratio that is about 1:3:7:90.

In certain embodiments, the MARC composition of the present disclosure is a “MARC6” composition. MARC6 compositions are designed for regeneration at or around the peripheral macula at about 3.0 mm and up to about 4.5 mm linear eccentricity of the inner and outer boundaries, respectively. In certain MARC6 compositions, the L:M ratio ranges from about 1.3:1 to about 2.8:1. In certain embodiments, the MARC6 L:M ratio is about 2:1. In certain embodiments, MARC6 compositions comprise about 6% to about 9% S-cones (as a percentage of total cones). In certain embodiments, MARC6 compositions comprise about 55% to about 85% rods (as a percentage of total cones+rods). In certain embodiments, MARC6 compositions have a S:M:L:R ratio that is about 2:9:19:70.

In certain embodiments, the MARC composition of the present disclosure is a “MARC7” composition. MARC7 compositions are designed for regeneration at or around the pericentric retina at about 4.5 mm and up to about 6.0 mm linear eccentricity of the inner and outer boundaries, respectively. In certain MARC7 compositions, the L:M ratio ranges from about 1.3:1 to about 2.8:1. In certain embodiments, the MARC7 L:M ratio is about 2:1. In certain embodiments, MARC7 compositions comprise about 6% to about 9% S-cones (as a percentage of total cones). In certain embodiments, MARC7 compositions comprise about 50% to about 80% rods (as a percentage of total cones+rods). In certain embodiments, MARC7 compositions have a S:M:L:R ratio that is about 2:11:22:65.

In certain embodiments, the MARC composition of the present disclosure is a “MARC8” composition. MARC8 compositions are designed for regeneration at or around the peripheral retina at about 6.0 mm and up to about 7.5 mm linear eccentricity of the inner and outer boundaries, respectively. In certain MARC8 compositions, the L:M ratio ranges from about 1.3:1 to about 2.8:1. In certain embodiments, the MARC8 L:M ratio is about 2:1. In certain embodiments, MARC8 compositions comprise about 7% to about 10% S-cones (as a percentage of total cones). In certain embodiments, MARC8 compositions comprise about 50% to about 80% rods (as a percentage of total cones+rods). In certain embodiments, MARC8 compositions have a S:M:L:R ratio that is about 3:10:22:65.

In certain embodiments, the MARC composition of the present disclosure is a “MARC9” composition. MARC9 compositions are designed for regeneration at or around the far peripheral retina at about 7.5 mm or higher linear eccentricity. In certain MARC9 compositions, the L:M ratio ranges from about 1.3:1 to about 2.8:1. In certain embodiments, the MARC9 L:M ratio is about 2:1. In certain embodiments, MARC9 compositions comprise about 7% to about 10% S-cones (as a percentage of total cones). In certain embodiments, MARC9 compositions comprise about 60% to about 90% rods (as a percentage of total cones+rods). In certain embodiments, MARC9 compositions have a S:M:L:R ratio that is about 2:7:14:75.

In certain embodiments, the MARC designs will be combined to straddle one or more contiguous or non-contiguous zones. For example, but not limitation, the present disclosure is directed to MARC compositions comprising a population of cone cells, wherein the ratio of S, M, and L cones falls within particular ranges in one region of the composition and in another region of the composition, the ratio of S, M, and L cones falls within another particular range. Such design variations in MARC can be created such that regenerative substrates can be designed to target degenerative loci straddling one or more contiguous or non-contiguous antero-posterior eccentricities. The MARC variation targeting each target location can be designed to mimic the S:M:L:R ratio that occurs naturally in trichromatic patients with normal color vision at the targeted eccentricities.

In certain embodiments, the MARC compositions of the present disclosure can comprise 1, 2, 3, 4, 5, 6, 7, 8, or 9 different regions, where each region can be designed to mimic the S:M:L:R ratio that occurs naturally in trichromatic patients with normal color vision at a targeted eccentricities. For example, but not limitation, a MARC composition can comprise a region comprising a MARC1 composition and a region comprising a MARC2 composition. In certain embodiments, a MARC composition can comprise a region comprising a MARC2 composition and a region comprising a MARC3 composition. In certain embodiments, a MARC composition can comprise a region comprising a MARC3 composition and a region comprising a MARC4 composition. In certain embodiments, a MARC composition can comprise a region comprising a MARC4 composition and a region comprising a MARC5 composition. In certain embodiments, a MARC composition can comprise a region comprising a MARC5 composition and a region comprising a MARC6 composition. In certain embodiments, a MARC composition can comprise a region comprising a MARC6 composition and a region comprising a MARC7 composition. In certain embodiments, a MARC composition can comprise a region comprising a MARC7 composition and a region comprising a MARC8 composition. In certain embodiments, a MARC composition can comprise a region comprising a MARC8 composition and a region comprising a MARC9 composition.

In certain embodiments, a MARC composition can comprise a region comprising a MARC1 composition, a region comprising a MARC2 composition, a region comprising a MARC3 composition, a region comprising a MARC4 composition, a region comprising a MARC5 composition, a region comprising a MARC6 composition, a region comprising a MARC7 composition, a region comprising a MARC8 composition, and/or a region comprising a MARC9 composition. For example, but not by way of limitation, a MARC composition can comprise a region comprising a MARC1 composition, region comprising a MARC2 composition, and/or a region comprising a MARC3 composition. In certain embodiments, a MARC composition can comprise a region comprising a MARC1 composition, region comprising a MARC2 composition, a region comprising a MARC3 composition and/or a region comprising a MARC4 composition. In certain embodiments, a MARC composition can comprise a region comprising a MARC1 composition, region comprising a MARC2 composition, a region comprising a MARC3 composition, a region comprising a MARC4 composition and/or a region comprising a MARC5 composition. In certain embodiments, a MARC composition can comprise a MARC1 composition, region comprising a MARC2 composition, a region comprising a MARC3 composition, a region comprising a MARC4 composition, a region comprising a MARC5 composition and/or a region comprising a MARC6 composition. In certain embodiments, a MARC composition can comprise a region comprising a MARC1 composition, region comprising a MARC2 composition, a region comprising a MARC3 composition, a region comprising a MARC4 composition, a region comprising a MARC5, a region comprising a MARC6 composition and/or a region comprising a MARC7 composition. In certain embodiments, a MARC composition can comprise a region comprising a region comprising a MARC1 composition, region comprising a MARC2 composition, a region comprising a MARC3 composition, a region comprising a MARC4 composition, a region comprising a MARC5, a region comprising a MARC6, a region comprising a MARC7 composition and/or a region comprising a MARC8 composition. In certain embodiments, a MARC composition can comprise a region comprising a MARC1 composition, region comprising a MARC2 composition, a region comprising a MARC3 composition, a region comprising a MARC4 composition, a region comprising a MARC5, a region comprising a MARC6, a region comprising a MARC7, a region comprising a MARC8 composition and/or a region comprising a MARC9 composition.

In certain embodiments, the MARC can comprise discontinuous regions, e.g., a region comprising a MARC1 composition and region comprising a MARC3 composition. Additional non-limiting examples of such MARCs comprising discontinuous regions include: a MARC comprising a region comprising a MARC1 composition and a region comprising a MARC3, MARC4, MARC5, MARC6, MARC7, MARC8, or MARC9 composition; a MARC comprising a region comprising a MARC2 composition and a region comprising a MARC4, MARC5, MARC6, MARC7, MARC8, or MARC9 composition; a MARC comprising a region comprising a MARC3 composition and a region comprising a MARC1, MARC5, MARC6, MARC7, MARC8, or MARC9 composition; a MARC comprising a region comprising a MARC4 composition and a region comprising a MARC1, MARC2, MARC6, MARC7, MARC8, or MARC9 composition; a MARC comprising a region comprising a MARC5 composition and a region comprising a MARC1, MARC2, MARC3, MARC7, MARC8, or MARC9 composition; a MARC comprising a region comprising a MARC6 composition and a region comprising a MARC1, MARC2, MARC3, MARC4, MARC8, or MARC9 composition; a MARC comprising a region comprising a MARC7 composition and a region comprising a MARC1, MARC2, MARC3, MARC4, MARC5, or MARC9 composition; a MARC comprising a region comprising a MARC8 composition and a region comprising a MARC1, MARC1, MARC2, MARC3, MARC4, MARC5 or MARC6 composition; and a MARC comprising a region comprising a MARC9 composition and a region comprising a MARC1, MARC2, MARC3, MARC4, MARC5, MARC6, or MARC7 composition.

In certain embodiments, the MARC compositions of the present disclosure can comprise a population of cone cells, wherein one or more of the cone outer segments show a large capacitance. As used herein, MARC compositions wherein one or more of the cone outer segments show a large capacitance, refers to MARC compositions characterized by membrane currents in the range of 500-2500 pA.

### 5.3 Methods of Producing Macular Repair Cell Compositions

In one aspect, the present disclosure relates to the manufacture of MARC therapeutics. In certain embodiments, MARC can be generated via human retinal organoid culture. For example, but not limitation, MARC can be generated using unique stem cell based human retinal organoid protocols that specifically enrich for S, M, and/or L cones. By enriching for specific ratios of S, M, and/or L cones, the methods of manufacture described herein can produce MARC designed to regenerate the approximate cellular composition that is found at specific zones (or across specific zones) in normal trichromatic subjects.

For example, but not limitation, the present disclosure is directed to methods for inducing directed differentiation of cells into a population of macular repair cells by contacting the cells with one or more signaling molecules under conditions capable of directing the differentiation of the cells into a population of macular repair cells. In certain embodiments, the cells induced to differentiate into a population of macular repair cells are embryonic stem cells, induced nonembryonic pluripotent cells, or engineered pluripotent cells. In certain embodiments, the signaling molecules used to contact the cells induced to differentiate into a population of macular repair cells are thyroid hormone, retinoic acid, as well as combinations thereof.

In certain embodiments, the population of macular repair cells will be prepared from an organoid. For example, but not by way of limitation, organoids that find use in the preparation of populations of macular repair cells can be prepared in accordance with the following “General Organoid Differentiation Protocol.” As the basis for organoid preparation and differentiation one of skill in the art can select an appropriate embryonic stem cell (ESC) or induced pluripotent stem cell (iPSC), for example, but not by way of limitation, H7 WA07, H7iCas9 ESCs, or EP1.1 iPSCs. To aggregate, cells can then be passaged in Accutase (SCR005, Sigma), e.g., at 37° C. for about 12 min, to ensure complete dissociation. Cells can then be seeded in 50 μLs of mTeSR1 at 3,000 cells/well into 96-well ultra-low adhesion round bottom Lipidure coated plates (51011610, NOF) or ultra-low attachment microplate (7007, Corning), although alternative densities and containers are considered within the scope of the instant protocol. Cells can then be placed in hypoxic conditions (e.g., about 10% CO₂ and about 5% O₂) for about 24 hours to enhance survival. Cells will naturally aggregate by gravity over 24 hours.

On about day 1, cells can be moved to normoxic conditions (e.g., about 5% CO₂). On or about days 1-3, about 50 μLs of BE6.2 media, or other appropriate media, containing about 3 μM Wnt inhibitor (IWR1e: 681669, EMD Millipore) and about 1% (v/v) Matrigel can be added to each well. On or about days 4-9, about 100 μL of media can be removed from each well, and about 100 μL of media can be added. On or about days 4-5, BE6.2 media, or other appropriate media, containing about 3 μM Wnt inhibitor and about 1% Matrigel can be added. On or about days 6-7, BE6.2 media, or other appropriate media, containing about 1% Matrigel (354230, BD Biosciences) can be added. On or about days 8-9, BE6.2 media, or other appropriate media, containing about 1% Matrigel and about 100 nM Smoothened agonist (SAG: 566660, EMD Millipore) can be added.

On or about day 10, aggregates can be transferred to 15 mL tubes (or other acceptable container), rinsed about 3× in about 5 mL DMEM (11885084, Gibco) or other appropriate media, and resuspended in BE6.2, or other appropriate media, with about 100 nM SAG in untreated 10 cm polystyrene petri dishes, or other appropriate container. From this point on, media can be changed about every other day. Aggregates can be monitored and manually separated if stuck together or to the bottom of the plate.

On or about days 13-16, LTR media, or other appropriate media, with about 100 nM SAG can be added. On or about day 16, retinal vesicles can be manually dissected using, e.g., sharpened tungsten needles. After dissection, cells can be transferred into 15 mL tubes, or other acceptable containers, and washed about 2× with about 5 mL of DMEM or other acceptable media. On or about days 16-20, cells can be maintained in LTR, or other acceptable media, and washed about 2× with about 5 mL of DMEM, or other acceptable media, before being transferred to new plates, or other acceptable containers, to wash off dead cells. To increase survival and differentiation, about 1.04 UM all-trans retinoic acid (ATRA; R2625; Sigma) can be added to LTR medium, or other acceptable media, from about days 20-43. As noted below, additional time windows of exposure to about 1.04 UM all-trans retinoic acid can be added depending on the desired L or M cone cell composition. About 10 μM gamma-secretase inhibitor (DAPT, 565770, EMD Millipore) can be added to LTR, or other acceptable media, from about days 28-42. In certain embodiments, organoids can be grown at low density (about 10 to about 20 per 10 cm dish) to reduce aggregation. Periodically, organoids can be culled from the plate based on absence of clear laminal structure indicating proper retinal organoid growth.

In certain embodiments, the population of macular repair cells are enriched for S (blue) cones. For example, but not by way of limitation, the populations of macular repair cells enriched for S (blue) cones can be produced by the above-described General Organoid Differentiation Protocol, where the ESCs or iPSCs employed to generate the organoid comprise a Thrβ (Thrβ1 and Thrβ2) knockout. For example, but not by way of limitation, CRISPR/Cas9 can be used, e.g., in human ESCs, to delete a shared exon that codes for part of the DNA binding domain of Thrβ as described in detail in Eldred et al. A variety of other strategies, however, can be used to knockout Thrβ expression and would be within the scope of the currently disclosed subject matter.

In certain embodiments, the population of macular repair cells are enriched for M (green) cones. For example, but not by way of limitation, the populations of macular repair cells enriched for M (green) cones can be produced by the above-described General Organoid Differentiation Protocol, but where the organoid is further exposed to RA from about day 43 to about 130, which results in M (green) cone enriched populations of cells by about day 200.

In certain embodiments, the population of macular repair cells are enriched for L (red) cones. For example, but not by way of limitation, the populations of macular repair cells enriched for L (red) cones can be produced by the above-described General Organoid Differentiation Protocol, but where the organoid is further exposed to RA from about day 130 to about day 200 yielded L cone-enriched populations of cells at day 200.

In certain embodiments, the populations of macular repair cells described herein will regenerate the approximate cellular composition that is found at specific zones (or across specific zones) in normal trichromatic subjects via the mixture of an appropriate number of cone cells, prepared as described herein and combined to achieve the desired S-, M-, and L-cone ratios, further in combination with an appropriate number of rod cells to achieve the desired S:M:L:R ratio.

In certain embodiments, the populations of macular repair cells can be produced, e.g., in independent organoids, and then mixed to achieve the desired S-, M-, and L-cone ratios, further in combination with an appropriate number of rod cells to achieve the desired S:M:L:R ratio. For example, but not by way of limitation, Table 1 provides exemplary combinations of cell compositions (including thyroid hormone T3 concentration and retinoic acid concentration conditions used in their production and duration of culture) to produce the MARC1-MARC9 compositions described herein.

TABLE 1

| | | | | COMPONENT 1 | COMPONENT 2 | COMPONENT 3 |
|---|---|---|---|---|---|---|
| | S | | Illustrative | Interval of 20 nM | Interval of 2 μM | End day of 0 nM |
| | cones | | S:M:L:R | T3 treatment | retinoic acid treatment | T3 and 0 nM |
| MARC | (%) | Rod (R) % | ratio | [Start day - End day] | [Start day - End day] | RA treatment |
| MARC1 | 0-2 | 0 | 1:33:66:0 | 43-170 | Not used | 170 |
| MARC2 | 0-5 | 0 | 3:33:64:0 | 43-170 | 43-130 | 170 |
| MARC3 | 10-20 | 60 (55-80) | 6:11:23:60 | 43-200 | 43-130 | 230-250 |
| MARC4 | 7-10 | 80 (60-95) | 2:6:12:80 | 43-200 | 43-130 | 230-250 |
| MARC5 | 6-9 | 90 (70-95) | 1:3:7:90 | 43-200 | 43-130 | 250 |
| MARC6 | 6-9 | 70 (55-85) | 2:9:19:70 | 43-200 | 43-130 | 230-250 |
| MARC7 | 6-9 | 65 (50-80) | 2:11:22:65 | 43-200 | 43-130 | 230-250 |
| MARC8 | 7-10 | 65 (50-80) | 3:10:22:65 | 43-200 | 43-130 | 230-250 |
| MARC9 | 7-10 | 75 (60-90) | 2:7:14:75 | 43-200 | 43-130 | 250 |

Additional combinations, e.g., to produce compositions straddling one or more contiguous or non-contiguous zones, can be produced in similar fashion by producing a cell composition having the features of the first zone and pairing it with cells having the features of the second (or subsequent) zone.

In certain embodiments, the populations of macular repair cells described herein will be depleted of non-neuroretinal cells. For example, but not by way of limitation, such populations of macular repair cells can be depleted of forebrain-like cells, forebrain progenitor cells, and/or retinal pigment epithelial cells. Markers of forebrain-like cells and/or forebrain progenitor cells that can be used to verify depletion include, but are not limited to, one or more of NKX2.2, RGCC, NEUROD1, BTG2, GADD45A, and GADD45G. Markers of retinal pigment epithelial cells that can be used to verify depletion include, but are not limited to, one or more of BEST1, TIMP3, GRAMD3, and PITPNA.

### 5.4. Methods of Treatment Using Macular Repair Cell Compositions

In another aspect, the subject matter of the present disclosure is directed to macular regenerative therapy by MARC transplantation as a treatment to ameliorate functional deficits in people with retinal degenerative diseases. For example, but not limitation, retinal degenerative diseases that can be ameliorated by administration of the MARC compositions of the present disclosure include age-related macular degeneration, Stargardt disease, cone dystrophy, achromatopsia, Best disease, mitochondrial macular degeneration, pattern dystrophy, RDS-associated macular degeneration, and other forms of inherited macular dystrophy. As described herein, MARC therapeutics can be transplanted into the macula, and such transplantation can lead to regeneration of cone photoreceptor cells. Not only can MARC transplantation preserve and/or improve vision in people affected by macular diseases, but MARC delivery, as described in detail herein, is performed to ensure optimal maturation and integration of MARC therapeutics into the macula.

In certain embodiments, the method of the present disclosure are directed to treating age-related macular degeneration, Stargardt disease, cone dystrophy, achromatopsia, Best disease, mitochondrial macular degeneration, pattern dystrophy, RDS-associated macular degeneration, and other forms of inherited macular dystrophy comprising engraftment into the macula of a patient in need thereof of a macular repair cell composition comprising a population of cone cells, wherein the ratio of S, M, and L cones falls within range of ratios corresponding to each targeted location to thereby mimic the S:M:L:R ratio that occurs naturally in trichromatic patients with normal color vision at the targeted eccentricity.

In certain embodiments, the method of the present disclosure comprise engraftment into the macula of a patient in need thereof of a MARC1 composition. In certain embodiments, the methods comprise engraftment into the macula of a patient in need thereof of a MARC1 compositions designed for foveal center regeneration at or around the foveal umbo at about 0 mm and up to about 0.1 mm linear eccentricity of the inner and outer boundaries, respectively. In certain embodiments, the methods comprise engraftment into the macula of a patient in need thereof of a MARC1 composition where the L:M ratio ranges from about 1.3:1 to about 2.8:1. In certain embodiments, the MARC1 L:M ratio is about 2:1. In certain embodiments, the methods comprise engraftment into the macula of a patient in need thereof of a MARC1 composition that comprises up to about 2% S-cones (as a percentage of total cones). In certain embodiments, the methods comprise engraftment into the macula of a patient in need thereof of a MARC1 composition containing no rods. In certain embodiments, the methods comprise engraftment into the macula of a patient in need thereof of a MARC1 composition having a S:M:L:R ratio that is about 1:33:66:0.

In certain embodiments, the method of the present disclosure comprise engraftment into the macula of a patient in need thereof of a MARC2 composition. In certain embodiments, the methods comprise engraftment into the macula of a patient in need thereof of a MARC2 composition designed for foveal center regeneration at or around the foveola at about 0.1 mm and up to about 0.175 mm linear eccentricity of the inner and outer boundaries, respectively. In certain embodiments, the methods comprise engraftment into the macula of a patient in need thereof of a MARC2 composition where the L:M ratio ranges from about 1.3:1 to about 2.8:1. In certain embodiments, the MARC2 L:M ratio is about 2:1. In certain embodiments, the methods comprise engraftment into the macula of a patient in need thereof of a MARC2 composition comprising up to about 5% S-cones (as a percentage of total cones). In certain embodiments, the methods comprise engraftment into the macula of a patient in need thereof of a MARC2 composition containing no rods. In certain embodiments, the methods comprise engraftment into the macula of a patient in need thereof of a MARC2 composition having a S:M:L:R ratio that is about 3:33:64:0

In certain embodiments, the method of the present disclosure comprise engraftment into the macula of a patient in need thereof of a MARC3 composition. In certain embodiments, the methods comprise engraftment into the macula of a patient in need thereof of a MARC3 composition designed for foveal center regeneration at or around the fovea at about 0.175 mm and up to about 0.750 mm linear eccentricity of the inner and outer boundaries, respectively. In certain embodiments, the methods comprise engraftment into the macula of a patient in need thereof of a MARC3 composition where the L:M ratio ranges from about 1.3:1 to about 2.8:1. In certain embodiments, the MARC3 L:M ratio is about 2:1. In certain embodiments, the methods comprise engraftment into the macula of a patient in need thereof of a MARC3 composition comprising about 10% to about 20% S-cones cones (as a percentage of total cones). In certain embodiments, the methods comprise engraftment into the macula of a patient in need thereof of a MARC3 composition comprising about 55% to about 80% rods (as a percentage of total cones+rods). In certain embodiments, the methods comprise engraftment into the macula of a patient in need thereof of a MARC3 composition having a S:M:L:R ratio that is about 6:11:23:60.

In certain embodiments, the method of the present disclosure comprise engraftment into the macula of a patient in need thereof of a MARC4 composition. In certain embodiments, the methods comprise engraftment into the macula of a patient in need thereof of a MARC4 compositions designed for regeneration at or around the parafovea at about 0.750 mm and up to about 1.5 mm linear eccentricity of the inner and outer boundaries, respectively. In certain embodiments, the methods comprise engraftment into the macula of a patient in need thereof of a MARC4 composition where the L:M ratio ranges from about 1.3:1 to about 2.8:1. In certain embodiments, the MARC4 L:M ratio is about 2:1. In certain embodiments, the methods comprise engraftment into the macula of a patient in need thereof of a MARC4 composition comprising about 7% to about 10% S-cones (as a percentage of total cones). In certain embodiments, the methods comprise engraftment into the macula of a patient in need thereof of a MARC4 composition comprising about 60% to about 95% rods (as a percentage of total cones+rods). In certain embodiments, the methods comprise engraftment into the macula of a patient in need thereof of a MARC4 composition having a S:M:L:R ratio that is about 2:6:12:80.

In certain embodiments, the method of the present disclosure comprise engraftment into the macula of a patient in need thereof of a MARC5 composition. In certain embodiments, the methods comprise engraftment into the macula of a patient in need thereof of a MARC5 composition designed for regeneration at or around the perifovea at about 1.5 mm and up to about 3.0 mm linear eccentricity of the inner and outer boundaries, respectively. In certain embodiments, the methods comprise engraftment into the macula of a patient in need thereof of a MARC5 composition where the L:M ratio ranges from about 1.3:1 to about 2.8:1. In certain embodiments, the MARC5 L:M ratio is about 2:1. In certain embodiments, the methods comprise engraftment into the macula of a patient in need thereof of a MARC5 composition comprising about 6% to about 9% S-cones (as a percentage of total cones). In certain embodiments, the methods comprise engraftment into the macula of a patient in need thereof of a MARC5 composition comprising about 75% to about 95% rods (as a percentage of total cones+rods). In certain embodiments, the methods comprise engraftment into the macula of a patient in need thereof of a MARC5 composition having a S:M:L:R ratio that is about 1:3:7:90.

In certain embodiments, the method of the present disclosure comprise engraftment into the macula of a patient in need thereof of a MARC6 composition. In certain embodiments, the methods comprise engraftment into the macula of a patient in need thereof of a MARC6 composition designed for regeneration at or around the peripheral macula at about 3.0 mm and up to about 4.5 mm linear eccentricity of the inner and outer boundaries, respectively. In certain embodiments, the methods comprise engraftment into the macula of a patient in need thereof of a MARC6 composition where the L:M ratio ranges from about 1.3:1 to about 2.8:1. In certain embodiments, the MARC6 L:M ratio is about 2:1. In certain embodiments, the methods comprise engraftment into the macula of a patient in need thereof of a MARC6 composition comprising about 6% to about 9% S-cones (as a percentage of total cones). In certain embodiments, the methods comprise engraftment into the macula of a patient in need thereof of a MARC6 composition comprising about 55% to about 85% rods (as a percentage of total cones+rods). In certain embodiments, the methods comprise engraftment into the macula of a patient in need thereof of a MARC6 composition having a S:M:L:R ratio that is about 2:9:19:70.

In certain embodiments, the method of the present disclosure comprise engraftment into the macula of a patient in need thereof of a MARC7 composition. In certain embodiments, the methods comprise engraftment into the macula of a patient in need thereof of a MARC7 composition designed for regeneration at or around the pericentric retina at about 4.5 mm and up to about 6.0 mm linear eccentricity of the inner and outer boundaries, respectively. In certain embodiments, the methods comprise engraftment into the macula of a patient in need thereof of a MARC7 composition where the L:M ratio ranges from about 1.3:1 to about 2.8:1. In certain embodiments, the MARC7 L:M ratio is about 2:1. In certain embodiments, the methods comprise engraftment into the macula of a patient in need thereof of a MARC7 composition comprising about 6% to about 9% S-cones (as a percentage of total cones). In certain embodiments, the methods comprise engraftment into the macula of a patient in need thereof of a MARC7 composition comprising about 50% to about 80% rods (as a percentage of total cones+rods). In certain embodiments, the methods comprise engraftment into the macula of a patient in need thereof of a MARC7 composition having a S:M:L:R ratio that is about 2:11:22:65.

In certain embodiments, the method of the present disclosure comprise engraftment into the macula of a patient in need thereof of a MARC8 composition. In certain embodiments, the methods comprise engraftment into the macula of a patient in need thereof of a MARC8 composition designed for regeneration at or around the peripheral retina at about 6.0 mm and up to about 7.5 mm linear eccentricity of the inner and outer boundaries, respectively. In certain embodiments, the methods comprise engraftment into the macula of a patient in need thereof of a MARC8 composition where the L:M ratio ranges from about 1.3:1 to about 2.8:1. In certain embodiments, the MARC8 L:M ratio is about 2:1. In certain embodiments, the methods comprise engraftment into the macula of a patient in need thereof of a MARC8 composition comprising about 7% to about 10% S-cones (as a percentage of total cones). In certain embodiments, the methods comprise engraftment into the macula of a patient in need thereof of a MARC8 composition comprising about 50% to about 80% rods (as a percentage of total cones+rods). In certain embodiments, the methods comprise engraftment into the macula of a patient in need thereof of a MARC8 composition having a S:M:L:R ratio that is about 3:10:22:65.

In certain embodiments, the method of the present disclosure comprise engraftment into the macula of a patient in need thereof of a MARC9 composition. In certain embodiments, the methods comprise engraftment into the macula of a patient in need thereof of a MARC9 composition designed for regeneration at or around the far peripheral retina at about 7.5 mm or higher linear eccentricity. In certain embodiments, the methods comprise engraftment into the macula of a patient in need thereof of a MARC9 composition where the L:M ratio ranges from about 1.3:1 to about 2.8:1. In certain embodiments, the MARC9 L:M ratio is about 2:1. In certain embodiments, the methods comprise engraftment into the macula of a patient in need thereof of a MARC9 composition comprising about 7% to about 10% S-cones (as a percentage of total cones). In certain embodiments, the methods comprise engraftment into the macula of a patient in need thereof of a MARC9 composition comprising about 60% to about 90% rods (as a percentage of total cones+rods). In certain embodiments, the methods comprise engraftment into the macula of a patient in need thereof of a MARC9 composition having a S:M:L:R ratio that is about 2:7:14:75.

In certain embodiments, the methods comprise engraftment into the macula of a patient in need thereof of a MARC designed to straddle one or more contiguous or non-contiguous zones. For example, but not limitation, the present disclosure is directed methods that comprise engraftment into the macula of a patient in need thereof of a MARC composition comprising a population of cone cells, wherein the ratio of S, M, and L cones falls within particular ranges in one region of the composition and in another region of the composition, the ratio of S, M, and L cones falls within another particular range. Such design variations in MARC can be created such that regenerative substrates can be designed to target degenerative loci straddling one or more contiguous or non-contiguous antero-posterior eccentricities. The MARC variation targeting each target location can be designed to mimic the S:M:L:R ratio that occurs naturally in trichromatic patients with normal color vision at the targeted eccentricities.

In certain embodiments, the methods comprise engraftment into the macula of a patient in need thereof of a MARC composition comprising 1, 2, 3, 4, 5, 6, 7, 8, or 9 different regions, where each region can be designed to mimic the S:M:L:R ratio that occurs naturally in trichromatic patients with normal color vision at a targeted eccentricities. For example, but not limitation, a MARC composition for use in the methods described herein can comprise a region comprising a MARC1 composition and a region comprising a MARC2 composition. In certain embodiments, a MARC composition for use in the methods described herein can comprise a region comprising a MARC2 composition and a region comprising a MARC3 composition. In certain embodiments, a MARC composition for usc in the methods described herein can comprise a region comprising a MARC3 composition and a region comprising a MARC4 composition. In certain embodiments, a MARC composition for use in the methods described herein can comprise a region comprising a MARC4 composition and a region comprising a MARC5 composition. In certain embodiments, a MARC composition for use in the methods described herein can comprise a region comprising a MARC5 composition and a region comprising a MARC6 composition. In certain embodiments, a MARC composition for use in the methods described herein can comprise a region comprising a MARC6 composition and a region comprising a MARC7 composition. In certain embodiments, a MARC composition for use in the methods described herein can comprise a region comprising a MARC7 composition and a region comprising a MARC8 composition. In certain embodiments, a MARC composition for use in the methods described herein can comprise a region comprising a MARC8 composition and a region comprising a MARC9 composition.

In certain embodiments, a MARC composition for use in the methods described herein can comprise a region comprising a MARC1 composition, a region comprising a MARC2 composition, a region comprising a MARC3 composition, a region comprising a MARC4 composition, a region comprising a MARC5 composition, a region comprising a MARC6 composition, a region comprising a MARC7 composition, a region comprising a MARC8 composition, and/or a region comprising a MARC9 composition. For example, but not by way of limitation, a MARC composition for use in the methods described herein can comprise a region comprising a MARC1 composition, region comprising a MARC2 composition, and/or a region comprising a MARC3 composition. In certain embodiments, a MARC composition for use in the methods described herein can comprise a region comprising a MARC1 composition, region comprising a MARC2 composition, a region comprising a MARC3 composition and/or a region comprising a MARC4 composition. In certain embodiments, a MARC composition for use in the methods described herein can comprise a region comprising a MARC1 composition, region comprising a MARC2 composition, a region comprising a MARC3 composition, a region comprising a MARC4 composition and/or a region comprising a MARC5 composition. In certain embodiments, a MARC composition for use in the methods described herein can comprise a MARC1 composition, region comprising a MARC2 composition, a region comprising a MARC3 composition, a region comprising a MARC4 composition, a region comprising a MARC5 composition and/or a region comprising a MARC6 composition. In certain embodiments, a MARC composition for use in the methods described herein can comprise a region comprising a MARC1 composition, region comprising a MARC2 composition, a region comprising a MARC3 composition, a region comprising a MARC4 composition, a region comprising a MARC5, a region comprising a MARC6 composition and/or a region comprising a MARC7 composition. In certain embodiments, a MARC composition for use in the methods described herein can comprise a region comprising a region comprising a MARC1 composition, region comprising a MARC2 composition, a region comprising a MARC3 composition, a region comprising a MARC4 composition, a region comprising a MARC5, a region comprising a MARC6, a region comprising a MARC7 composition and/or a region comprising a MARC8 composition. In certain embodiments, a MARC composition for use in the methods described herein can comprise a region comprising a MARC1 composition, region comprising a MARC2 composition, a region comprising a MARC3 composition, a region comprising a MARC4 composition, a region comprising a MARC5, a region comprising a MARC6, a region comprising a MARC7, a region comprising a MARC8 composition and/or a region comprising a MARC9 composition.

In certain embodiments, the MARC composition for use in the methods described herein can comprise discontinuous regions, e.g., a region comprising a MARC1 composition and region comprising a MARC3 composition. Additional non-limiting examples of such MARC compositions for use in the methods described herein comprising discontinuous regions include: a MARC comprising a region comprising a MARC1 composition and a region comprising a MARC3, MARC4, MARC5, MARC6, MARC7, MARC8, or MARC9 composition; a MARC comprising a region comprising a MARC2 composition and a region comprising a MARC4, MARC5, MARC6, MARC7, MARC8, or MARC9 composition; a MARC comprising a region comprising a MARC3 composition and a region comprising a MARC1, MARC5, MARC6, MARC7, MARC8, or MARC9 composition; a MARC comprising a region comprising a MARC4 composition and a region comprising a MARC1, MARC2, MARC6, MARC7, MARC8, or MARC9 composition; a MARC comprising a region comprising a MARC5 composition and a region comprising a MARC1, MARC2, MARC3, MARC7, MARC8, or MARC9 composition; a MARC comprising a region comprising a MARC6 composition and a region comprising a MARC1, MARC2, MARC3, MARC4, MARC8, or MARC9 composition; a MARC comprising a region comprising a MARC7 composition and a region comprising a MARC1, MARC2, MARC3, MARC4, MARC5, or MARC9 composition; a MARC comprising a region comprising a MARC8 composition and a region comprising a MARC1, MARC1, MARC2, MARC3, MARC4, MARC 5 or MARC6 composition; and a MARC comprising a region comprising a MARC9 composition and a region comprising a MARC1, MARC2, MARC3, MARC4, MARC5, MARC6, or MARC7 composition.

In certain embodiments, MARC delivery in the context of the methods described herein is performed using a device for the access and delivery of cells or other materials to the subretinal space. For example, but not limitation, the device for MARC delivery in the context of the methods described herein can be a device as described in PCT Application PCT/US2019/045074 (WO2020028892), which is incorporated by reference herein in its entirety.

Briefly, devices for MARC delivery in the context of the methods described herein, such as those described in PCT Application PCT/US2019/045074 (WO2020028892), can access the subretinal space via the trans-scleral side. This is an ab externo approach. In certain embodiments, the cell delivery device can include two stacked layers, surrounded by a flexible outer surface, e.g., as illustrated in FIGS. 1-8 of PCT Application PCT/US2019/045074 (WO2020028892). In certain embodiments, the device can be configured to be flexible, such that it conforms to the natural curvature of the eye as it is advanced to the subretinal space. In certain embodiments, after the device is in place, the flexible outer surface is configured to protect the delicate tissue of the retina and retinal pigment epithelium and choroid, while there is also easy passage of the material or cells to be delivered between the two stacked layers.

In certain embodiments, a device for use in the context of the methods described herein can include an optical coherence tomography sensor directly integrated into a guide needle to allow for visualization of the subretinal space during the subretinal space opening process. In certain embodiments, a device for use in the context of the methods described herein can comprise a flexible cannula and injector system to safely navigate the propagation tunnel. In certain embodiments, a device for use in the context of the methods described herein can comprise a plunger system which exert force against the MARC composition with reduced or eliminated risk of damaging it.

### 6. EXAMPLES

The following examples are merely illustrative of the presently disclosed subject matter and should not be considered as limitations in any way.

### Example 1: Preparation of S Vs L/M Cone Enriched Organoids

As described in detail in Eldred et al., Science 2018, 362:6411 (2018):eaau6348, (Eldred et al.) which is incorporated herein by reference in its entirety, human organoids can recapitulate the specification of cone subtypes observed in the human retina, including the temporal generation of S cones followed by L and M cones. Moreover, this regulation is controlled by thyroid hormone signaling, which is necessary and sufficient to control cone subtype fates through the nuclear hormone receptor thyroid hormone receptor β (Thrβ). As described in Eldred et al. and outlined below, the instant example provides exemplary methods to prepare S vs L/M cone enriched organoids.

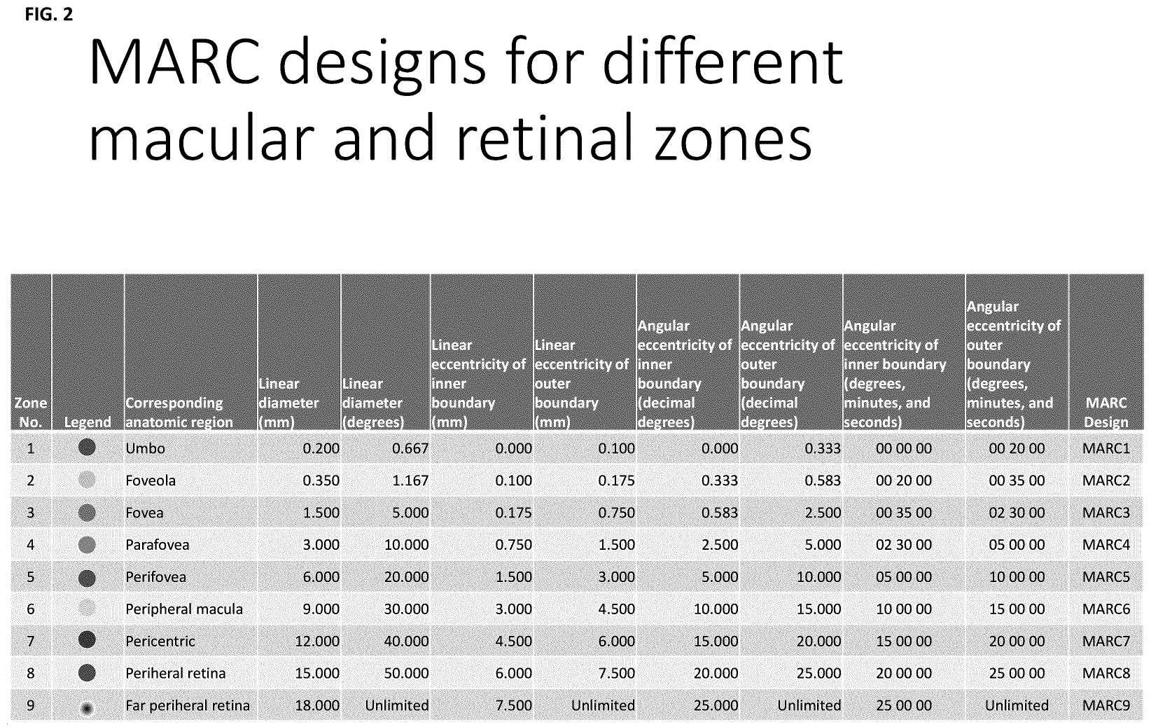

To determine if the specification of cone cells in organoids recapitulates development in the human retina, Eldred et al. compared features of cone subtypes in human organoids with those of adult retinal tissue. Adult human retinas and organoids at day 200 of differentiation displayed similar ratios of S to L/M cones as indicated by expression of S- or L/M-opsins (adult, S=13%, L/M=87%; organoid, S=29%, L/M=71%) (FIGS. 1, B and C, and FIG. S1A of Eldred et al.). Eldred et al. indicates that the difference in the ratios is likely due to the immaturity of the organoid at ˜6 months compared with the terminally differentiated adult retina. Eldred et al. also examined L/M cones with an antibody that recognizes both L- and M-opsin proteins because of their extremely high similarity. Both S and L/M cones expressed the cone-rod-homeobox transcription factor (CRX), a critical transcription factor for photoreceptor differentiation (FIGS. 2, A and E of Eldred et al.), indicating proper fate specification in organoids. Additionally, Eldred et al. showed that cones in organoids and retinas displayed similar morphologies, with L/M cones that had longer outer segments and wider inner segments than those of S cones (FIGS. 2, B to D and F to H of Eldred et al.). The outer segments of cones were also shown to be shorter in organoids than in adult retinas, which is consistent with postnatal maturation (FIGS. 2, D and H of Eldred et al.). Thus, Eldred et al. noted that cone subtypes in human retinal organoids displayed distributions, gene expression patterns, and morphologies similar to those of cones of the human retina.

Eldred et al. also examined the developmental dynamics of cone subtype specification in organoids. In the human retina, S cones are generated during fetal weeks 11 to 34 (days 77 to 238), whereas L/M cones are specified later, during fetal weeks 14 to 37 (days 98 to 259). Eldred et al. tracked the ratios and densities of S and L/M cones in organoids by means of antibody staining over 360 days of differentiation. Cones expressing S-opsin were first observed at day 150 (FIGS. 2, I, L, and M of Eldred et al.). The density of S cones leveled off at day 170 (FIG. 2M of Eldred et al.), at the time point when cones expressing L/M-opsin began to be observed (FIG. 2, J to M of Eldred et al.). The population of L/M cones increased dramatically until day 300 (FIG. 2, K to M of Eldred et al.), when they reached a steady-state density. The 20-day difference between S- and L/M-opsin expression onset in retinal organoids is similar to the 20-day difference observed in the appearance of S and L/M cones in the fetal retina. These observations show a temporal switch from S cone specification to L/M cone specification during retinal development.

Eldred et al. next conducted RNA sequencing (RNA-seq) through 250 days of induced pluripotent stem cell (iPSC)-derived organoid development. Eldred et al. found that S-opsin RNA was expressed first at day 111 and leveled off at day 160, whereas L/M-opsin RNA was expressed at day 160 and remained steady after day 180, which is consistent with the timeline of photoreceptor maturation in organoids and fetal retinas (FIG. 2N and FIG. S1B of Eldred et al.). Moreover, CRX RNA and CRX protein were expressed before opsins in organoids, which is similar to human development (FIG. 2N and FIG. S1, B to G of Eldred et al.). Thus, human organoids recapitulate many aspects of the developmental timeline of cone subtype specification observed in human retinas, providing a model system with which to uncover the mechanisms of these developmental changes.

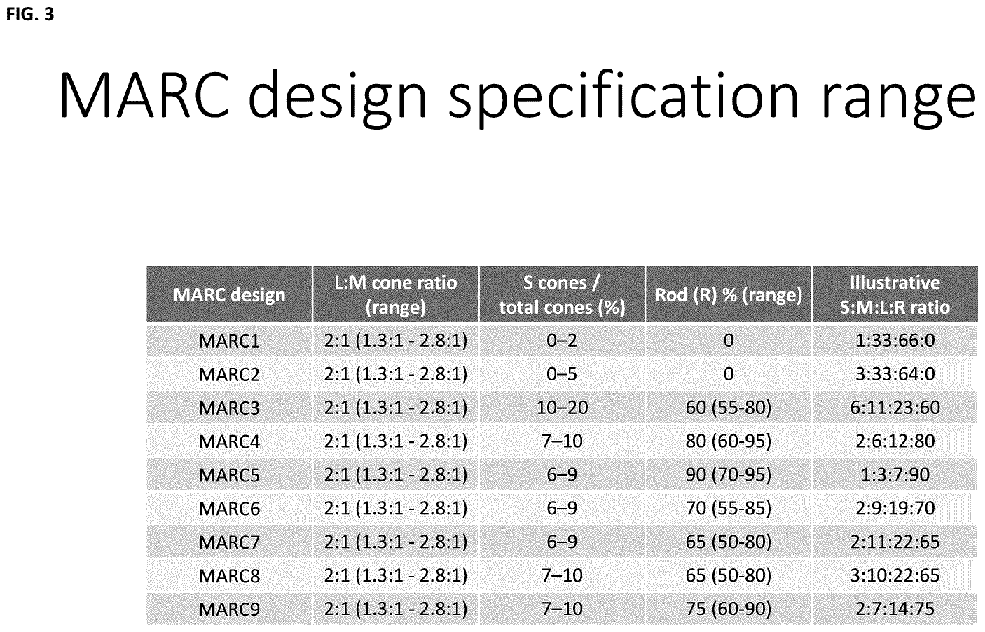

To directly test the role of Thrβ2 in human cone subtype specification, Eldred et al. used CRISPR/Cas9 in human embryonic stem cells (ESCs) to generate a homozygous mutation that resulted in early translational termination in the first exon of Thrβ2 (FIG. S2A of Eldred et al.). Surprisingly, organoids derived from these mutant stem cells displayed no differences in cone subtype ratio from genotypically wild-type organoids [wild type, S=62%, L/M=38%; Thrβ2 knockout (KO), S=59%, L/M=41%; P=0.83]. The S-to-L/M ratio is high for both wild-type controls and Thrβ2 KO organoids, likely owing to variability in organoid differentiation. Thus, unlike previous suggestions based on other species, Eldred et al. consider Thrβ2 dispensable for cone subtype specification in humans (FIG. 3, A to C of Eldred et al.).

Because Thrβ2 alone is not required for human cone subtype specification, Eldred et al. asked whether Thrβ1 and Thrβ2 together are required for cone subtype specification in humans. To completely ablate Thrβ function (Thrβ1 and Thrβ2), Eldred et al. used CRISPR/Cas9 in human ESCs to delete a shared exon that codes for part of the DNA binding domain of Thrβ (FIG. S2A of Eldred et al.). Thrβ null mutant retinal organoids displayed a complete conversion of all cones to the S subtype (wild type, S=27%, L/M=73%; Thrβ KO, S=100%, L/M=0%; P<0.0001) (FIGS. 3, D to E and H of Eldred et al.). In these mutants, all cones expressed S-opsin and had the S cone morphology (FIGS. 3, I and J of Eldred et al.). Thus, Thrβ is required to activate L/M and to repress S cone fates in the human retina.

As noted in Eldred et al., Thrβ binds with high affinity to triiodothyronine (T3), the more active form of thyroid hormone, to regulate gene expression. In addition, depletion or addition of T3 alters the ratios of S to M cones in rodents. Because L/M cones differentiate after S cones, Eldred et al. hypothesized that T3 acts through Thrβ late in retinal development to induce L/M cone fate and repress S cone fate. One prediction of this hypothesis is that addition of T3 early in development will induce L/M fate and repress S fate. To test this model, Eldred et al. added 20 nM T3 to ESC- and iPSC-derived organoids starting from days 20 to 50 and continued until day 200 of differentiation. Eldred et al. observed a dramatic conversion of cone cells to L/M fate (wild type, S=27%, L/M=73%; wild type+T3, S=4%, L/M=96%; P<0.01) (FIGS. 3, F and H, and FIG. S2B of Eldred et al.). Thus, early addition of T3 is sufficient to induce L/M fate and suppress S fate.

To test whether T3 acts specifically through Thrβ to control cone subtype specification, Eldred et al. differentiated Thrβ mutant organoids with early T3 addition. Thrβ mutation completely suppressed the effects of T3, generating organoids with only S cones (wild type+T3, S=4%, L/M=96%; Thrβ KO+T3, S=100%, L/M=0%; P<0.0001) (FIG. 3, F to H of Eldred et al.). Based on these results, Eldred et al. concluded that T3 acts though Thrβ to promote L/M cone fate and suppress S cone fate.

Eldred et al. also confirmed the regulation of L/M-opsin expression through thyroid hormone signaling in a retinoblastoma cell line, which expresses L/M-opsin when treated with T3 (FIGS. S2, C and D of Eldred et al.). T3-induced activation of L/M-opsin expression was suppressed upon RNA interference knockdown of Thrβ (FIGS. S2, E and F of Eldred et al.), which is similar to the suppression observed in human organoids.

Eldred et al. also demonstrated that, in organoids, early T3 addition not only converted cone cells to L/M fate but also dramatically increased cone density (FIGS. 3, F and K of Eldred et al.). Moreover, T3 acts specifically through Thrβ to control cone density (FIGS. 3, G and K of Eldred et al.). Early T3 addition may increase cone density by advancing and extending the temporal window of L/M cone generation.

Together, these results demonstrate that T3 signals though Thrβ to promote L/M cone fate and repress S cone fate in developing human retinal tissue.

### Materials & Methods as Described by Eldred et al.

Cell lines. H7 ESC (WA07, WiCell) and episomal-derived EP1.1 iPSC lines were used for differentiation, although, as described above in Section 5.3, other suitable cell lines are known in the art. WERI-Rb1 retinoblastoma cells were obtained from ATCC. Cell maintenance and organoid differentiation protocols are described in the supplementary materials of Eldred et al.

CRISPR mutations. All mutations were generated in H7 ESCs. Cells were modified to express an inducible Cas9 element. Plasmids for guide RNA (gRNA) transfection were generated by using the pSpCas9(BB)-P2A-Puro plasmid modified from the pX459_V2.0 plasmid (62988, Addgene) by replacing T2A with a P2A sequence. Mutations were confirmed with polymerase chain reaction sequencing. Gene diagrams of deletions are displayed in FIG. S2A of Eldred et al. Detailed transfection procedures, gRNA sequences, and homology arm sequences are included in the supplementary materials of Eldred et al.

Immunohistochemistry. Primary antibodies were used in Eldred et al. at the following dilutions: goat anti-SW-opsin (1:200 for organoids, 1:500 for human retinas) (Santa Cruz Biotechnology), rabbit anti-LW/MW-opsins (1:200 for organoids, 1:500 for human retinas) (Millipore), mouse anti-CRX (1:500) (Abnova), and mouse anti-Rhodopsin (1:500) (GeneTex). All secondary antibodies were Alexa Fluor-conjugated (1:400) and made in donkey (Molecular Probes). Detailed methods for fixation, microscopy, and image processing of organoids, retinas, and WERI-Rb1 cells are included in the supplementary materials of Eldred et al.

Organoid Age. Opsin expression time course. EP1 iPSC-derived organoids for time course experiments were binned in Eldred et al. into 10-day increments for analysis. Organoids were binned into day 130 [actual day 129 (n=3 organoids)], day 150 [actual day 152 (n=4 organoids)], day 170 [actual day 173 (n=2 organoids)], day 200 [actual days 194 to 199 (n=7 organoids)], day 290 [actual day 291 (n=3 organoids)], and day 360 [actual day 361 (n=3 organoids)]. Quantifications of outer-segment lengths and inner-segment widths were measured in day 361 organoids (n=3 organoids).

Opsin expression in different conditions. iCas9 H7 ESC-derived organoids for Thrb2 KOs and controls were analyzed in Eldred et al. at day 200. Organoids for Thrb KO, control, and wild-type+T3 were analyzed in Eldred et al. at two time points: two organoids were taken at day 199 for each group, and one was taken at day 277 for each group. T3-treated organoids were taken at time points between day 195 and day 200 for different differentiations. For each treatment group and genotype, organoids were compared with control organoids grown in parallel.

RNA-seq time course. EP1 iPSC-derived organoids were analyzed in Eldred et al. at time points ranging from day 10 to day 250 of differentiation. Eldred et al. took samples at day 10 (n=3 organoids), day 20 (n=2 organoids), day 35 (n=3 organoids), day 69 (n=3 organoids), day 111 (n=3 organoids), day 128 (n=3 organoids), day 158 (n=2 organoids), day 173 (n=3 organoids), day 181 (n=3 organoids), day 200 (n=3 organoids), and day 250 (n=3 organoids). RNA from individual organoids was extracted by using the Zymo Direct-zol RNA Microprep Kit (Zymo Research) according to manufacturer's instructions. Libraries were prepared in Eldred et al. using the Illumina TruSeq stranded mRNA kit and sequenced on an Illumina NextSeq 500 with single 200-base pair reads.

RNA-seq Time Course Analysis. Expression levels were quantified in Eldred et al. using Kallisto (version 0.34.1) with the following parameters: “-b 100-1 200-s 10-t 20-single”. The Gencode release 28 comprehensive annotation was used as the reference transcriptome. Transcripts per million (TPM) values (table S1 of Eldred et al.) were then used to generate graphs in Prism and heatmaps in R by using ggplot2. The distributions of transcripts were plotted so as to identify the best low TPM cutoff (FIG. S5A of Eldred et al.). The threshold was determined to be 0.7 log(TPM+1)-5 TPM- and this value was used as an inflection point for the heatmaps. Heatmaps for FIG. S3, A to C of Eldred et al., were made similarly, by using CPM values from Hoshino et al., Dev. Cell 43, 763-779.c4 (2017).

Measurements and Quantification. Measurements of retinal arca and cell morphology in Eldred et al. were done by using ImageJ software. Quantifications and statistics (except for RNA-seq data) in Eldred et al. were done in GraphPad Prism, with a significance cutoff of 0.01. Statistical tests are listed in figure legends of Eldred et al. and all error bars represent the SEM.

### Example 2: Preparation of L Vs M Cone Enriched Organoids

As described in detail in Hadyniak et al., bioRxiv, 2021.03.30.437763 (2021) (Hadyniak et al.) which is incorporated herein by reference in its entirety, human organoids can recapitulate the specification of cone subtypes observed in the human retina, including the temporal generation of L and M cones. Moreover, this regulation is controlled by retinoic acid (RA) signaling. As described in Hadyniak et al. and outlined below, the instant example provides exemplary methods to prepare L vs M cone enriched organoids.

As described in Hadyniak et al., RA signaling early promotes M cone fate and suppresses L cone fate in human retinal organoids. Differentiation of human retinal organoids involves addition of all-trans RA (hereafter referred to as RA) on days 20-43 to promote early retinal patterning (FIG. S5 of Hadyniak et al.). RA was not added from day 43 to day 200, a timeframe which includes the end of primitive retina differentiation and the complete duration of cell fate specification (‘No RA’).

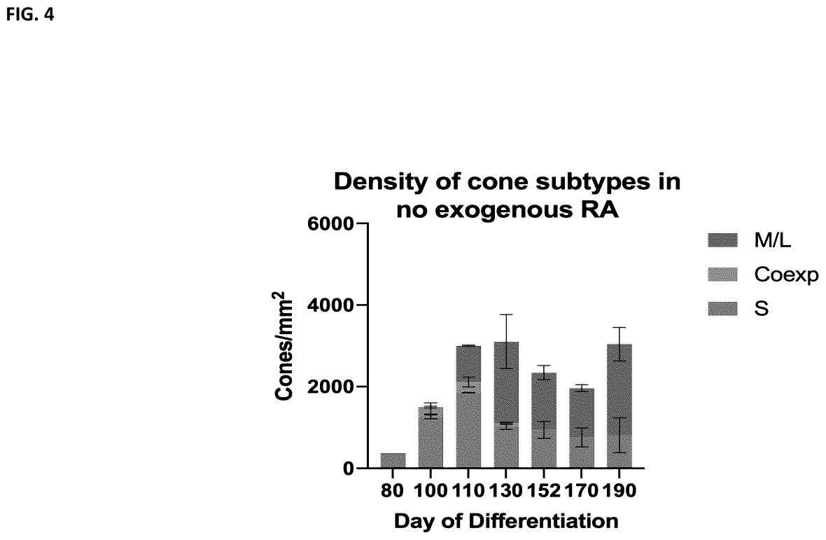

Hadyniak et al. first used an in situ hybridization approach to examine the timing of M and L cone generation during human retinal organoid development. At day 120, Hadyniak et al. observed very few M and L cones (FIG. 4A, S6A of Hadyniak et al.). Hadyniak et al. first observed significant numbers of M and L cones on day 140 (FIG. 4A, S6A of Hadyniak et al.). From day 140 through day 200, organoids were enriched for L cones (FIG. 4A-B, S6A of Hadyniak et al.). These observations suggested that human retinal organoids differentiated with this protocol are missing developmental cues to generate large populations of M cones before L cones as in human fetal development.

To test whether addition of RA induced M cone generation in retinal organoids, Hadyniak et al. added 1.0 μM RA over different timeframes and assessed M and L cones at day 200 (FIG. 4C of Hadyniak et al.). Organoids grown in supplemental RA throughout development failed to differentiate, yielding minimal M or L cones (N=6). Addition of RA early in development from days 43 to 130 yielded organoids with almost exclusively M cones at day 200 (98.35% M, 1.65% L, 0% co-expressing; FIG. 4D-E of Hadyniak et al.; ‘Early RA’). To corroborate this observation, Hadyniak et al. conducted RNA-seq on ESC-derived organoids grown in ‘Early RA’ conditions and observed high M-opsin and minimal L-opsin expression (FIG. S1C of Hadyniak et al.). Hadyniak et al. also analyzed previously published RNA-seq data on iPSC-derived organoids grown in ‘Early RA’ conditions and observed near exclusive expression of M-opsin (FIG. S1D of Hadyniak et al.). Addition of RA late in development from days 130 to 200 yielded L cone-enriched organoids at day 200 (6.35% M, 92.56% L, 1.10% co-expressing; FIG. 4D-E of Hadyniak et al.; ‘Late RA’), similar to organoids grown with no additional RA. Hadyniak et al. observed no significant differences in overall M/L cone densities at day 200 across experimental conditions (FIG. S6B of Hadyniak et al.). Together, these data indicate that RA is sufficient to induce M cones and suppress L cones early in retinal organoid development

### Materials & Methods as Described by Hadyniak et al.

Cell line maintenance. H7 ESC (WA07, WiCell) and episomal-derived EP1.1 iPSC were used in Hadyniak et al. for retinal organoid differentiation. Stem cells were maintained in Hadyniak et al. in mTeSR™ (85857, Stem Cell Technologies) on 1% (v/v) Matrigel-GFR™ (354230, BD Biosciences) coated dishes and grown at 37° C. in a HERAcell 150i or 160i 10% CO₂ and 5% O2 incubator (Thermo Fisher Scientific). Cells were passaged in Hadyniak et al. every 4-5 days according to confluence as in Wahlin et al., Sci Rep 7, 766 (2017), Cells were passaged with Accutase (SCR005, Sigma) for 7-12 minutes to be dissociated to single cells. Cells in Accutase were added 1:2 to mTeSR™1 plus 5 μM Blebbistatin (Bleb, B0560, Sigma), pelleted at 150 g for 5 minutes, and suspended in mTeSR™1 plus Bleb and plated at 5,000-15,000 cells per well in a 6-well plate. Cells were fed with mTeSR™ 48 hours following passage and every 24 hours until passaged again. To minimize cell stress, no antibiotics were used.

Weri-Rb-1 retinoblastoma cells were obtained from ATCC and maintained in Hadyniak et al. in RPMI 1640 Medium (11875135, Gibco)+10% Fetal Bovine Serum (16140071, Gibco)+1× Penicillin-Streptomycin (30-002-CI, Corning) at 37° C. in a HERAcell 150i or 160i 5% CO₂ incubator (Thermo Fisher Scientific). Cells were passaged every 4 days at ˜1×10⁵-2×10⁶ cells/mL in uncoated flasks by pelleting at 150 g for 5 minutes and resuspending in fresh media.

Cell lines were tested monthly for mycoplasma using MycoAlert (LT07, Lonza).

Cell Culture Media. Stem Cell media mTeSR1 (85857, StemCell Technologies). E6 supplement: 970 μg/mL Insulin (11376497001, Roche), 535 μg/mL holo-transferrin (T0665, Sigma), 3.20 mg/mL L-ascorbic acid (A8960, Sigma), 0.7 μg/mL sodium selenite (S5261, Sigma). BE6.2 media for early retinal differentiation: 2.5% E6 supplement (above), 2% B27 Supplement (50×) minus Vitamin A (12587010, Gibco), 1% Glutamax (35050061, Gibco), 1% NEAA (11140050, Gibco), 1 mM Pyruvate (11360070, Gibco), and 0.87 mg/mL NaCl in DMEM (11885084, Gibco). LTR (Long-Term Retina) media: 25% F12 (11765062, Gibco) with 2% B27 Supplement (50×) (17504044, Gibco), 10% heat inactivated FBS (16140071, Gibco), 1 mM Sodium Pyruvate, 1% NEAA, 1% Glutamax and 1 mM taurine (T-8691, Sigma) in DMEM (11885084, Gibco). RPMI+supplement media: 10% heat inactivated FBS (16140071, Gibco), 2.5% penicillin (30-002-CI, Corning) in RPMI Medium 1640 (11875135, Gibco).

Retinoic acid treatment: For organoids, 1.04 UM all-trans retinoic acid (ATRA; R2625; Sigma) in LTR.

Thyroid hormone treatment: For Weri-Rb1 cells, 100 nM T3 (T6397, Sigma) in RPMI+supplement media.

Organoid differentiation. Organoids were differentiated from H7 WA07, H7iCas9 ESCs, or EP1.1 iPSCs as described in Eldred et al. 2018 with minor variations (FIG. S5 of Hadyniak et al.). Pluripotent stem cells were well-maintained. Cultures with minimal to no spontaneous differentiation were used for aggregation. To aggregate, cells were passaged in Accutase (SCR005, Sigma) at 37° C. for 12 min to ensure complete dissociation. Cells were seeded in 50 μLs of mTeSR1 at 3,000 cells/well into 96-well ultra-low adhesion round bottom Lipidure coated plates (51011610, NOF) or ultra-low attachment microplate (7007, Corning). Cells were placed in hypoxic conditions (10% CO₂ and 5% O2) for 24 hours to enhance survival. Cells naturally aggregated by gravity over 24 hours.

On day 1, cells were moved to normoxic conditions (5% CO₂). On days 1-3, 50 μLs of BE6.2 media containing 3 μM Wnt inhibitor (IWR1e: 681669, EMD Millipore) and 1% (v/v) Matrigel were added to each well. On days 4-9, 100 μLs of media were removed from each well, and 100 μLs of media were added. On days 4-5, BE6.2 media containing 3 μM Wnt inhibitor and 1% Matrigel was added. On days 6-7, BE6.2 media containing 1% Matrigel (354230, BD Biosciences) was added. On days 8-9, BE6.2 media containing 1% Matrigel and 100 nM Smoothened agonist (SAG: 566660, EMD Millipore) was added.

On day 10, aggregates were transferred to 15 mL tubes, rinsed 3× in 5 mL DMEM (11885084, Gibco), and resuspended in BE6.2 with 100 nM SAG in untreated 10 cm polystyrene petri dishes. From this point on, media was changed every other day. Aggregates were monitored and manually separated if stuck together or to the bottom of the plate.

On days 13-16, LTR media with 100 nM SAG was added. On day 16, retinal vesicles were manually dissected using sharpened tungsten needles. After dissection, cells were transferred into 15 mL tubes and washed 2× with 5 mLs of DMEM. On days 16-20, cells were maintained in LTR and washed 2× with 5 mLs of DMEM, before being transferred to new plates to wash off dead cells. To increase survival and differentiation, 1.04 μM all-trans retinoic acid (ATRA; R2625; Sigma) was added to LTR medium from days 20-43. Additional time windows of 1.04 μM were added depending on experimental conditions. 10 μM gamma-secretase inhibitor (DAPT, 565770, EMD Millipore) was added to LTR from days 28-42. Organoids were grown at low density (10-20 per 10 cm dish) to reduce aggregation. Periodically, organoids were culled from the plate based on absence of clear laminal structure indicating proper retinal organoid growth.

RNA-Seq experiments. EP1 iPSC-derived organoids were previously grown and analyzed for Eldred et al. at time points ranging from day 10 to day 250 of differentiation. Samples were taken at day 10 (N=3), day 20 (N=2), day 35 (N=3), day 69 (N=3), day 111 (N=3), day 128 (N=3), day 158 (N=2), day 173 (N=3), day 181 (N=3), day 200 (N=3), and day 250 (N=3).

H7 ESC-derived organoids were taken at day 329 (N=3).

Weri-Rb-1 samples were grown in control RPMI+supplement media or T3-treated RPMI+supplement media for four days (N=1).

RNA from individual samples was extracted using the Zymo Direct-zol RNA Microprep Kit (R2062, Zymo Research) according to manufacturer's instructions. Libraries were prepared using the Illumina TruSeq stranded mRNA kit and sequenced on an Illumina NextSeq 500 with single 75 bp reads.

Human retina and organoid preparation and cryosectioning. Human retina. Donor sample was flash frozen on dry ice 10.9 hours postmortem and stored at −80 C. The human eye was allowed to come to room temperature in 1×PBS and the retina was dissected out of the eye. The retina was fixed for 45 minutes in 10% neutral buffered formalin (HT501128, Sigma) and washed in 1×PBS. Subsections of retina were mounted in Tissue-Tek O.C.T. compound (4583, Sakura), placed on dry ice to freeze, and stored at −80 C. The retina was sectioned in 10 μm sections. Slides were air dried for 6 hours to overnight with a post-fixation step of 15 minutes in 10% neutral buffered formalin (HT501128, Sigma) and washed in 1×PBS. Slides were dried and stored at −80 C for less than 3 months before use.

Organoid. Organoids were fixed for 45 minutes in 10% neutral buffered formalin (HT501128, Sigma) and washed in 1×PBS. Organoids were placed in a 25% sucrose in 0.1 M phosphate buffer solution overnight, and then mounted in Tissue-Tek O.C.T. compound (4583, Sakura), placed on dry ice to freeze, and stored at −80 C. Organoids were sectioned in 10 μm sections. Slides were air dried for 6 hours to overnight with a post-fixation step of 15 minutes in 10% neutral buffered formalin (HT501128, Sigma) and washed in 1×PBS. Slides were dried and stored at −80 C for less than 3 months before use.

Organoid cell lines employed in Hadyniak et al.: No RA: H7 ESC (N=3); Early RA: H7 ESC (N=1), H7 iCas9 ESC (N=2); and Late RA: H7iCas9 ESC (N=5).

RNA in situ hybridization. BaseScope RNA in situ hybridization was performed according to manufacturer's instructions and modified with several changes. Probe sequences were designed by ACD Biotechne based on OPNIMW and OPNILW mRNA sequences NM_000513.2 NM_020061.5 from the human genome hg38.

Sections were allowed to come to room temperature from storage at −80 C and rehydrated in 1×PBS. Samples were pretreated according to manufacturer's instructions: 10 minutes of RNAscope Hydrogen Peroxide followed by a wash in dH₂O and then 2 washes in 1×PBS.

RNAscope Protease III was applied at a 1:15 dilution in 1×PBS for 15 minutes in a humid chamber for HEK293 cells. RNAscope Protease IV was applied for 20 minutes in a humid chamber for organoids and human eye samples. Samples were washed in 1×PBS twice.

Probes were added at the manufacturer suggested concentration to samples in the HybEZ Humidity Control rack with lid and insert into the HybEZ oven for 2 hours at 40° C. Samples were washed twice in 1× RNAscope wash buffer for 2 min.

Amplification and development washes were done in Hadyniak et al. with the manufacturer reagents without change to the recommended concentrations. All washes were done in the HybEZ Humidity Control rack with lid and insert, either at room temperature (RT) or at 40 C in the HybEZ oven.

2×2 min washes in 1× RNAScope Buffer were conducted between each reagent wash. The final wash was in tap water. Slides were baked for a minimum of 30 minutes at 65 C in the HybEZ oven. Samples were preserved with VectaMount (Vector Laboratories, H-5000) mounting media and a sealed coverslip.

Imaging Organoids. All serially sectioned organoids were imaged and counted manually. Organoids that had fewer than 150 cones (n≤150) were removed from analysis in Hadyniak et al. Statistical tests are listed in figure legends of Hadyniak et all and all error bars represent SEM.

### Example 3: Temporal Variation in the Ratio of Photoreceptor Identity in Culture Conditions Lacking Retinoic Acid (RA)

Retinal organoids were grown, generally as described in Examples 1 and 2, in media lacking addition of exogenous RA following day 43 (end of early retinal development). The organoids were stained for S-opsin and M/L-opsin at various timepoints over development. From this data the density of cone subtypes was quantified over time. As illustrated in FIG. 4, implementation of the instant protocol results in S cones being specified first, comprising 100% of the cone cells. When M/L cone specification begins, a subpopulation of cones coexpress S and M/L opsin. This co-expression is later resolved and over time M/L cones makeup the majority of the cone subtypes. At day 130, more than 50% of cones are of the M/L identity and this approximate ratio persists at further timepoints studied.

The contents of all figures and all references, patents and published patent applications and Accession numbers cited throughout this application are expressly incorporated herein by reference.

What is claimed is:

1. A cellular composition comprising a population of L cones and M cones, wherein the ratio of L cones to M cones (L:M) falls within a predetermined range, wherein the predetermined L:M range is from about 1.3:1 to about 2.8:1.

2. The cellular composition of claim 1, wherein the predetermined L:M ratio is about 2:1.

3. The cellular composition of claim 1, wherein:
a) the population of cones comprises up to about 2% S cones as a percentage of total cones and the ratio of the population of S, M, and L cones (S:M:L) is about 1:33:66; or
b) the population of cones comprises up to about 5% S cones as a percentage of total cones and the S:M:L is about 3:33:64.

4. The cellular composition of claim 1, wherein:
a) the particular retinal eccentricity is at or around the foveal umbo; or
b) the particular retinal eccentricity inner boundary is at about 0 mm and the outer boundary is at about 0.1 mm linear eccentricity.

5. The cellular composition of claim 1, wherein:
a) the particular retinal eccentricity is at or around the foveal center; or
b) the particular retinal eccentricity inner boundary is at about 0.1 mm and the outer boundary is at about 0.175 mm linear eccentricity.

6. The cellular composition of claim 1, wherein the composition comprises rods (“R”) and wherein the S:M:L:R ratio falls within a predetermined range and the predetermined S:M:L:R ratio is a ratio that occurs at a particular retinal eccentricity.

7. The cellular composition of claim 6, wherein:
a) the particular retinal eccentricity is at or around the foveal center; or
b) the particular retinal eccentricity inner boundary is at about 0.175 mm and the outer boundary is at about 0.750 mm linear eccentricity.

8. The cellular composition of claim 6, wherein:
a) the population of cones comprises about 10% to about 20% S cones as a percentage of total cones; or
b) the population of rods comprises about 55% to about 80% rods as a percentage of total cones and rods combined and the ratio of S:M:L:R is about 6:11:23:60.

9. The cellular composition of claim 6, wherein:
a) the particular retinal eccentricity is at or around the parafovea; or
b) the particular retinal eccentricity inner boundary is at about 0.750 mm and the outer boundary is at about 1.50 mm linear eccentricity.

10. The cellular composition of claim 6, wherein:
a) the population of cones comprises about 7% to about 10% S cones as a percentage of total cones; or
b) the population of cones comprises about 6% to about 9% S cones as a percentage of total cones.

11. The cellular composition of claim 6, wherein:
a) the population of rods comprises about 60% to about 95% rods as a percentage of total cones and rods combined, wherein the ratio of S:M:L:R is about 2:6:12:80;
b) the population of rods comprises about 75% to about 95% rods as a percentage of total cones and rods combined and the ratio of S:M:L:R is about 1:3:7:90;
c) the population of rods comprises about 55% to about 85% rods as a percentage of total cones and rods combined and the ratio of S:M:L:R is about 2:9:19:70;
d) the population of rods comprises about 50% to about 80% rods as a percentage of total cones and rods combined and the ratio of S:M:L:R is about 2:11:22:65 or about 3:10:22:65; or
e) the population of rods comprises about 60% to about 90% rods as a percentage of total cones and rods combined and the ratio of S:M:L:R is about 2:7:14:75.

12. The cellular composition of claim 6, wherein:
a) the particular retinal eccentricity is at or around the perifovea; or
b) the particular retinal eccentricity inner boundary is at about 1.50 mm and the outer boundary is at about 3.0 mm linear eccentricity.

13. The cellular composition of claim 6, wherein:
a) the particular retinal eccentricity is at or around the peripheral macula; or
b) the particular retinal eccentricity inner boundary is at about 3.0 mm and the outer boundary is at about 4.5 mm linear eccentricity.

14. The cellular composition of claim 6, wherein:
a) the particular retinal eccentricity is at or around the pericentric retina; or
b) the particular retinal eccentricity inner boundary is at about 4.50 mm and the outer boundary is at about 6.0 mm linear eccentricity.

15. The cellular composition of claim 6, wherein:
a) the particular retinal eccentricity is at or around the peripheral retina; or
b) the particular retinal eccentricity inner boundary is at about 6.0 mm and the outer boundary is at about 7.5 mm linear eccentricity.

16. The cellular composition of claim 6, wherein:
a) the particular retinal eccentricity is at or around the far peripheral retina; or
b) the particular retinal eccentricity inner boundary is at about 7.50 mm and the outer boundary is greater than about 7.50 mm linear eccentricity.

17. The cellular composition of claim 1, wherein:
a) the composition comprises two regions wherein each region comprises a different M:L ratio;
b) the composition comprises two regions wherein each region comprises a different S:M:L ratio; or
c) the composition comprises two regions wherein each region comprises a different S:M:L:R ratio.

18. A cellular composition comprising population of cones, wherein one or more of the cone outer segments exhibits a capacitance, wherein the composition is characterized by membrane currents in the range of 500-2500 pA.

19. A method of treating age-related macular degeneration or retinal degeneration comprising engraftment into the macula of a patient in need thereof of a cellular composition comprising a population of L cones and M cones, wherein the ratio of L cones to M cones (L:M) falls within a predetermined range, wherein the predetermined L:M range is from about 1.3:1 to about 2.8:1.

20. The method of claim 19, wherein the retinal degeneration is due to age-related macular degeneration, Stargardt disease, cone dystrophy, achromatopsia, Best disease, mitochondrial macular degeneration, pattern dystrophy, or RDS-associated macular degeneration.
